(19) United States
(12) Patent Application Publication
(10) Pub. No.: US 2023/0167417 A1
(43) Pub. Date: Jun. 1, 2023

(54) MODEL-BASED CONTROL FOR COLUMN-BASED CONTINUOUS VIRAL INACTIVATION OF BIOPHARMACEUTICALS

(71) Applicant: Massachusetts Institute of Technology, Cambridge, MA (US)

(72) Inventors: Richard Braatz, Cambridge, MA (US); Amos Lu, Cambridge, MA (US); Moo Sun Hong, Cambridge, MA (US)

(21) Appl. No.: 17/997,429

(22) PCT Filed: Apr. 30, 2021
(86) PCT No.: PCT/US2021/030148

§ 371 Date: Oct. 28, 2022

Related U.S. Application Data
(60) Provisional application No. 63/129,899, filed on Dec. 23, 2020.
(60) Provisional application No. 63/017,935, filed on Apr. 30, 2020.

(51) Int. Cl. C12N 7/00; C12M 1/00; C12M 1/42; C12M 1/36; C12M 1/34

(52) U.S. Cl. CPC C12N 7/00; C12M 47/12; C12M 35/08; C12M 41/48; C12M 41/26

(57) ABSTRACT

Provided herein is a column-based continuous viral inactivation system, comprising one or both of a pH feedback controller to adjust feed flow rates and a minimum residence time (MRT) feedback controller to adjust feed flow rates. Methods of viral inactivation with the system are also provided.

### CROSS-REFERENCE TO RELATED APPLICATIONS

This application claims the benefit of U.S. Provisional Application Ser. No. 63/017,935, filed Apr. 30, 2020, and U.S. Provisional Application Serial No. 63/129,899, filed Dec. 23, 2020, the entire disclosures of which are incorporated herein by reference.

### STATEMENT REGARDING FEDERALLY SPONSORED RESEARCH OR DEVELOPMENT

This invention was made with Government support under Grant No. U01 FD006483 awarded by the Food and Drug Administration. The Government has certain rights in the invention.

### BACKGROUND

The continuous manufacturing of biologics has been of interest to academia and industry due to reduced costs, increased flexibility, ease of standardization and scale up, and improvements in product quality [1, 2]. The complexity of these processes brings with them control challenges and opportunities [3,4]. Biologics derived from mammalian sources are expected to undergo two orthogonal virus removal processes in order to remove adventitious viruses or retroviruses that may be present in the master cell bank [5-7]. One common processing step is a batch low-pH hold [8] to inactivate enveloped viruses [9]. Attempts to adapt this technique to continuous processing have involved cyclic batch operation [10], continuous flow tubular reactors [11-15], or continuous flow column-based reactors [16, 17].

### SUMMARY

This Summary introduces a selection of concepts in simplified form that are described further below in the Detailed Description. This Summary neither identifies key or essential features, nor limits the scope, of the claimed subject matter.

None of the continuous flow reactors directly address the control challenges with operating a system as described herein. The operating pH and the residence time distribution are important process parameters in determining viral clearance and impact to product quality from over-incubation or excessive pH adjustment.

Herein is described a low-cost, column-based, continuous viral inactivation system constructed with off-the-shelf components. A fast and accurate model-based, pH feedback control scheme allows for rapid startup and effective disturbance rejection. The residence time distribution (RTD) is estimated periodically during operation through inverse tracer experiments and used to estimate minimum residence time (MRT), which in turn is used to adjust feed flow rates. Controlled validation experiments demonstrate its performance in pH and MRT setpoint tracking and feed buffer and column residence time disturbance rejection. Viral clearance testing demonstrates tight control of logarithmic reduction values (LRV) over extended operation.

In one aspect, the disclosure provides a column-based continuous viral inactivation system, comprising a pH feedback controller to adjust feed flow rates.

In another aspect, the disclosure provides a column-based continuous viral inactivation system, comprising a minimum residence time (MRT) feedback controller to adjust feed flow rates.

In another aspect, the disclosure provides a column-based continuous viral inactivation system, comprising a pH feedback controller and a minimum residence time (MRT) feedback controller to adjust feed flow rates.

In certain embodiments, the feedback controllers estimate residence time distribution (RTD) periodically during operation to adjust feed flow rates.

In certain embodiments, the feedback controllers estimate RTD at least once during operation to adjust feed flow rates.

In certain embodiments, the feedback controllers estimate RTD 1, 2, 3, 4, 5, 6, 7, 8, 9, 10, or more times during operation to adjust feed flow rates.

In certain embodiments, the RTD is estimated by injecting a tracer. In certain embodiments, the tracer is UV-transparent.

In certain embodiments, the MRT is about 1 minute to about 30 minutes. In certain embodiments, the MRT is 2.5 minutes, about 4 minutes, about 5 minutes, about 10 minutes, about 15 minutes, or about 20 minutes.

In certain embodiments, the system comprises: a first in-line mixer; a first pH electrode; a first UV absorbance sensor; a column; a second UV absorbance sensor; a second in-line mixer; and a second pH electrode; wherein each of the first in-line mixer, column, and second in-line mixer comprise an inlet, an outlet, and a tubular flow path.

In certain embodiments, the first in-line mixer mixes an acid solution and a fluid sample comprising one or more target molecules, thereby producing a pH-reduced fluid sample.

In certain embodiments, the pH-reduced fluid sample comprises a pH of about 2.0 to about 5.0. In certain embodiments, the pH-reduced fluid sample comprises a pH of 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

In certain embodiments, the first pH electrode and second pH electrode provide the pH to the pH feedback controller.

In certain embodiments, the pH-reduced fluid sample is incubated in the column for a time sufficient to inactivate one or more viruses in the pH-reduced fluid sample.

In certain embodiments, at least about 99.5% of the pH-reduced fluid sample by volume is incubated in the column for about 1 minute to about 120 minutes.

In certain embodiments, the column comprises an inert material.

In certain embodiments, the inert material comprises polymethyl methacrylate (PMMA), polyethylene, polypropylene, polyvinylchloride, and/or silica glass,

In certain embodiments, the column comprises a serpentine column.

In certain embodiments, the one or more target molecules comprise a protein, a carbohydrate, a polynucleotide, a lipid, a vitamin, or an antibiotic.

In certain embodiments, the one or more target molecules comprise an antibody.

In certain embodiments, the fluid sample comprises a protein A chromatography eluate comprising an antibody.

In certain embodiments, the protein A chromatography eluate comprises a pH of about 2.0 to about 5.0. In certain embodiments, the protein A chromatography eluate comprises a pH of 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

In certain embodiments, the second in-line mixer mixes a base solution and the pH-reduced fluid sample, thereby producing a pH-rebalanced fluid sample.

In certain embodiments, the pH-rebalanced fluid sample comprises a pH of about 6.5 to about 8.5. In certain embodiments, the pH-rebalanced fluid sample comprises a pH of about 7.0 to about 8.0.

In another aspect, the disclosure provides a column-based continuous viral inactivation system, comprising a minimum residence time (MRT) sufficient to inactivate a substantial portion of viruses in a fluid sample, wherein the MRT is maintained by periodically injecting a tracer into the system.

In certain embodiments, the MRT comprises a time for about a 0.01 fraction, 0.005 fraction, 0.001 fraction, 0.0005 fraction, 0.0001 fraction, 0.00005, or a 0.00001 fraction of a volume of the fluid sample to leave the system. In certain embodiments, the MRT comprises a time for about a 0.005 fraction of a volume of fluid to leave the system.

In certain embodiments, the MRT comprises about 1 minute to about 60 minutes. In certain embodiments, the MRT comprises about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or about 60 minutes.

In certain embodiments, the system comprises an MRT sufficient to inactivate about 95%, about 96%, about 97%, about 98%, about 99%, about 99.5%, about 99.6%, about 99.7%, about 99.8%, about 99.9%, or 100% of viruses in a fluid sample.

In certain embodiments, the tracer injected at least once during operation to maintain the MRT. In certain embodiments, the tracer injected 1, 2, 3, 4, 5, 6, 7, 8, 9, 10, or more times during operation to maintain the MRT.

In certain embodiments, the tracer is UV-transparent. In certain embodiments, the tracer is deionized water.

In one aspect, the disclosure provides a method for inactivating one or more viruses in a fluid sample comprising a target molecule in a column-based continuous viral inactivation system, wherein the method comprises: a) mixing the fluid sample with an acid to lower pH in a first mixing unit; b) continuously transferring the fluid sample from step a) to an inert column for incubation at a specified residence time to inactivate viruses; c) periodically adding a protein-free injection solution to the fluid sample before the fluid sample is transferred to the column; d) continuously transferring the fluid sample from step b) to a second mixing unit; e) mixing the fluid sample with a base in the second mixing unit; f) using pH electrodes to measure the pH of the fluid sample after the fluid sample flows through the first and second mixing units, wherein the pH electrodes provide feedback to control acid and base flow; g) monitoring fluid pulse before and after the fluid sample flows through the column by measuring UV absorbance at 280 nm, wherein the monitoring characterizes the residence time distribution of the column in real-time; and h) adjusting total fluid flow in the column-based continuous viral inactivation system to maintain minimum residence time; wherein the viruses are inactivated.

In certain embodiments, the residence time distribution (RTD) is estimated periodically during operation by injecting a tracer into the system.

In certain embodiments, the residence time distribution (RTD) is estimated periodically during operation by injecting a tracer into the system and used to estimate minimum residence time (MRT), which in turn is used to adjust feed flow rates.

In certain embodiments, the tracer is UV-transparent. In certain embodiments, the tracer is deionized water.

In certain embodiments, the feedback controllers estimate RTD at least once during operation to adjust feed flow rates. In certain embodiments, the feedback controllers estimate RTD 1, 2, 3, 4, 5, 6, 7, 8, 9, 10, 11, 12, 13, 14, 15, 16, 17, 18, 19, 20, or more times during operation to adjust feed flow rates.

In certain embodiments, the MRT is about 1 minute to about 30 minutes. In certain embodiments, the MRT is 2.5 minutes, about 4 minutes, about 5 minutes, about 10 minutes, about 15 minutes, or about 20 minutes.

In certain embodiments, the MRT comprises a time for about a 0.01 fraction, 0.005 fraction, 0.001 fraction, 0.0005 fraction, 0.0001 fraction, 0.00005, or a 0.00001 fraction of a volume of the fluid sample to leave the system. In certain embodiments, the MRT comprises a time for about a 0.005 fraction of a volume of fluid to leave the system.

In certain embodiments, the MRT comprises about 1 minute to about 60 minutes. In certain embodiments, the MRT comprises about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or about 60 minutes.

In certain embodiments, the system comprises an MRT sufficient to inactivate about 95%, about 96%, about 97%, about 98%, about 99%, about 99.5%, about 99.6%, about 99.7%, about 99.8%, about 99.9%, or 100% of viruses in a fluid sample.

In certain embodiments, the pH is lowered to a pH of about 2.0 to about 5.0. In certain embodiments, the pH is lowered to a pH of 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

In certain embodiments, at least about 99.5% of the fluid sample by volume is incubated in the inert column for about 1 minute to about 120 minutes.

In certain embodiments, the inert column comprises polymethyl methacrylate (PMMA), polyethylene, polypropylene, polyvinylchloride, and/or silica glass,

In certain embodiments, the column comprises a serpentine column.

In certain embodiments, the target molecule comprises a protein, a carbohydrate, a polynucleotide, a lipid, a vitamin, or an antibiotic.

In certain embodiments, the target molecule comprises an antibody.

In certain embodiments, the fluid sample comprises a protein A chromatography eluate comprising an antibody.

The following Detailed Description references the accompanying drawings which form a part this application, and which show, by way of illustration, specific example implementations. Other implementations may be made without departing from the scope of the disclosure.

### BRIEF DESCRIPTION OF THE DRAWINGS

FIG. 1A illustrates a schematic of the column-based viral inactivation unit.

FIG. 1B illustrates a photo of the column-based viral inactivation unit.

FIG. 2A illustrates results of a pH controller setpoint tracking test.

FIG. 2B illustrates results of an MRT controller setpoint tracking test.

FIG. 2C illustrates results of absorbance measurements at the inlet and outlet of the column, overlaid with the RTD model.

FIG. 3A illustrates results of a pH controller disturbance rejection test.

FIG. 3B illustrates results of an MRT controller disturbance rejection test.

FIG. 4A-FIG. 4E depict viral inactivation with respect to pH. FIG. 4A illustrates the flow rates (mL/min) determined by the controllers. The lines, from top to bottom, correspond to Input, Base, Acid, and Injection. Injection corresponds to the injection of deionized water into the system to estimate RTD. FIG. 4B illustrates results absorbance measurements at the inlet and outlet of the column, overlaid with the RTD model. FIG. 4C illustrates the estimated MRT. FIG. 4D illustrates pH measurements over time. The top horizontal line corresponds to the second mixing unit. The bottom stepwise horizontal line corresponds to the first mixing unit. The black dashed lines indicate setpoints. The 5 vertical dotted lines represent the time when outlet from the system was sampled for the phage plaque assay. The pH setpoint for first mixing unit was changed from 3.5 to 3.8, 4.2, 4.5, and 4.8. The pH setpoint for the second mixing unit was 7.3 and MRT setpoint was 5 minutes. FIG. 4E illustrates the logarithmic reduction values (LRVs) of the samples from various pH setpoints for the first mixing unit. The horizontal dotted line shows the limit of detection (no live virus was detected for values on the horizontal dotted line).

FIG. 5A-FIG. 5E depict viral inactivation with respect to MRT. FIG. 5A illustrates the flow rates (mL/min) determined by the controllers. The lines, from top to bottom, correspond to Input, Base, Acid, and Injection. Injection corresponds to the injection of deionized water into the system to estimate RTD. FIG. 5B illustrates results absorbance measurements at the inlet and outlet of the column, overlaid with the RTD model. FIG. 5C illustrates pH measurements. FIG. 5D illustrates estimated MRT. The black dashed line indicates setpoints. The 5 vertical dotted lines represent the time when outlet from the system was sampled for the phage plaque assay. The MRT setpoint was changed from 20 minutes to 15, 10, 5, and 2.5 minutes. The pH setpoint for the first mixing unit was 4.5 and the pH setpoint for the second mixing unit was 7.3. FIG. 5E illustrates the LRVs of the samples from various MRT setpoints for the first mixing unit. The horizontal dotted line shows the limit of detection (no live virus was detected for values on the horizontal dotted line).

FIG. 6A-FIG. 6E depict viral inactivation while operating at a pH setpoint of 4.5 for the first mixing unit, a pH setpoint of 7.3 for the second mixing unit, and MRT setpoint of 15 minutes. FIG. 6A illustrates the flow rates (mL/min) determined by the controllers. The lines, from top to bottom, correspond to Base, Input, Acid, and Injection. Injection corresponds to the injection of deionized water into the system to estimate RTD. FIG. 6B illustrates results absorbance measurements at the inlet and outlet of the column, overlaid with the RTD model.

FIG. 6C illustrates pH measurements. The top line corresponds to the second mixing unit and the bottom line corresponds to the first mixing unit. FIG. 6D illustrates estimated MRT. The black dashed line indicates setpoints. The 5 vertical dotted lines represent the time when outlet from the system was sampled for the phage plaque assay. FIG. 6E illustrates the LRVs of the samples from various operating times.

FIG. 7 depicts a plot showing how the pH setpoint would be changed with respect to the total flow rate through the column for LRV_{Sp}=5, η=0.005, ν=4 [mL], pH₀=5.0, and

$$k = 0.7 [ \frac{1}{min} ] ,$$

where the parameters ν, pH₀, and k are obtained from experimental data in FIG. 4-FIG. 6 of the disclosure.

FIG. 8 depicts a plot showing flow rates chosen by the controller for an extended operation to confirm the repeatability of the viral inactivation for the alternative inlet/input flowrate fixed by the upstream process to be 0.26 mL/min.

### DETAILED DESCRIPTION

Reference numbers in brackets “[ ]” herein refer to the corresponding literature listed in the attached Bibliography which forms a part of this Specification, and the literature is incorporated by reference herein.

Herein is described a low-cost, column-based continuous viral inactivation system constructed with off-the-shelf components. A fast and accurate model-based pH feedback control scheme allows for rapid startup and effective disturbance rejection. The residence time distribution (RTD) is estimated periodically during operation through inverse tracer experiments and used to estimate minimum residence time (MRT), which in turn is used to adjust feed flow rates. Controller validation experiments demonstrate its performance in pH and MRT setpoint tracking and feed buffer and column residence time disturbance rejection. Viral clearance testing demonstrates tight control of logarithmic reduction values (LRV) over extended operation.

As used herein, the term “minimum residence time” or “MRT” refers to the minimum time in which a set volume is incubated. For example, MRT can refer to the minimum time in which about 99.5% of the fluid in a system is incubated at low pH. Alternatively, MRT can refer to the time in which a fraction or percent of volume or fluid leaves the system. This fraction may vary from 0.005 to 10¹. For example, a MRT for a fraction of 0.005 (0.5%) is the time for that fraction (or percent) of fluid to leave the system (e.g., the column-based continuous viral inactivation system).

As used herein, the term “residence time distribution” or “RTD” refers to a frequency distribution of times that different fractions of a fluid (e.g., a fluid sample comprising one or more target molecules) spends within a system. The average of all the times for a fluid corresponds to the mean residence time.

In certain embodiments, the RTD in the column-based continuous viral inactivation system is estimated periodically.

In certain embodiments, the RTD in the column-based continuous viral inactivation system is estimated at least once during the operation of the system.

In certain embodiments, the RTD in the column-based continuous viral inactivation system is estimated 1, 2, 3, 4, 5, 6, 7, 8, 9, 10, or more times during the operation of the system.

In certain embodiments, the RTD in the column-based continuous viral inactivation system is estimated 1, 2, 3, 4, 5, 6, 7, 8, 9, 10, 11, 12, 13, 14, 15, 16, 17, 18, 19, or 20 times during the operation of the system.

In certain embodiments, the RTD in the column-based continuous viral inactivation system is estimated about every 5 minutes, about every 10 minutes, about every 15 minutes, about every 20 minutes, about every 25 minutes, about every 30 minutes, about every 35 minutes, about every 40 minutes, about every 45 minutes, about every 50 minutes, about every 55 minutes, about every 60 minutes, about every 65 minutes, about every 70 minutes, about every 75 minutes, about every 80 minutes, about every 85 minutes, about every 90 minutes, about every 95 minutes, about every 100 minutes, about every 105 minutes, about every 110 minutes, about every 115 minutes, or about every 120 minutes, during the operation of the system.

In certain embodiments, the feedback controllers estimate the RTD during operation to adjust feed flow rates.

In certain embodiments, the RTD is estimated by injecting a tracer into the system. A “tracer” as used herein, is an inert or non-reactive compound that is capable of being detected in the system. Detection can be direct, by measuring the tracer compound itself, or indirect, by measuring a reduction in a signal (e.g., an A₂₂₀ signal from a UV detector) due to dilution in the system. A tracer measured indirectly is referred to as a “negative tracer” or “inverse tracer”. In certain embodiments, the tracer is UV-transparent. In certain embodiments, the tracer is deionized water. The tracer is injected into an inlet of the system and is detected one or more times as it passes through the system. Exemplary tracers include, but are not limited to, a protein-free water solution, an acetone water solution, a dye, a radioactive compound, nanoparticles (e.g., gold nanoparticles), riboflavin, or dextrose. Further description of tracers and their use to determine RTD is disclosed in Amarikwa et al. (Biotechnol J. 2019. 14(2):e1700726).

As used herein, the term “pH feedback controller” or “pH controller” refers to a device that monitors pH in a system (e.g., a fluid system, such as a column-based continuous viral inactivation system). Said controller is capable of adjusting the flow rate of said system with a pump (e.g., a peristaltic pump) and/or introduce acid or base to adjust the pH in the system to a setpoint.

As used herein, the term “minimum residence time (MRT) feedback controller” or “MRT controller” refers to a device that monitors the MRT in a system (e.g., a fluid system, such as a column-based continuous viral inactivation system). Said controller is capable of adjusting the flow rate of said system with a pump (e.g., a peristaltic pump) to adjust the MRT in the system to a setpoint. For example, but in no way limiting, the MRT feedback controller can be set to a MRT of 0.005 (i.e., the time that a fraction of 0.005 of the volume of fluid leaves the system), and adjust the flow rate of the system when the detected MRT is above or below 0.005.

In certain embodiments, the MRT comprises a time for about a 0.01 fraction, 0.005 fraction, 0.001 fraction, 0.0005 fraction, 0.0001 fraction, 0.00005, or a 0.00001 fraction of a volume of fluid to leave the system (e.g., column-based continuous viral inactivation system).

In certain embodiments, the MRT comprises a time for about a 0.005 fraction of a volume of fluid to leave the system (e.g., column-based continuous viral inactivation system).

In certain embodiments, the MRT is about 1 minute to about 60 minutes. In certain embodiments, the MRT is about 1 minute to about 30 minutes. In certain embodiments, the MRT is about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or about 60 minutes.

In certain embodiments, the MRT is 2.5 minutes, about 4 minutes, about 5 minutes, about 10 minutes, about 15 minutes, or about 20 minutes.

In certain embodiments, the system comprises: a first in-line mixer; a first pH electrode; a first UV absorbance sensor; a column; a second UV absorbance sensor; a second in-line mixer; and a second pH electrode; wherein each of the first in-line mixer, column, and second in-line mixer comprise an inlet, an outlet, and a tubular flow path.

In certain embodiments, the first in-line mixer mixes an acid solution and a fluid sample comprising one or more target molecules, thereby producing a pH-reduced fluid sample.

In certain embodiments, the pH-reduced fluid sample comprises a pH of about 2.0 to about 5.0. In certain embodiments, the pH-reduced fluid sample comprises a pH of 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In certain embodiments, the first pH electrode and second pH electrode provide the pH to the pH feedback controller.

In certain embodiments, the pH-reduced fluid sample is incubated in the column for a time sufficient to inactivate one or more viruses in the pH-reduced fluid sample. In certain embodiments, the pH-reduced fluid sample is incubated in the column for a time sufficient to inactivate about 80%, about 85%, about 90%, about 95%, about 96%, about 97%, about 98%, about 99%, or 100% of the viruses in the pH-reduced fluid sample.

In certain embodiments, the pH-reduced fluid sample is incubated in the column for a time sufficient to achieve a logarithmic reduction value (LRV) of about 10, about 9, about 8, about 7, about 6, about 5, about 4, about 3, about 2, about 1, or 0.

In certain embodiments, at least about 90% of the pH-reduced fluid sample by volume is incubated in the column for about 1 minute to about 120 minutes. In certain embodiments, at least about 90% of the pH-reduced fluid sample by volume is incubated in the column for about 1 minute, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes.

In certain embodiments, about 90%, about 95%, about 96%, about 97%, about 98%, about 99%, about 99.5%, about 99.6%, about 99.7%, about 99.8%, about 99.9%, or 100% of the pH-reduced fluid sample by volume is incubated in the column for about 1 minute, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes.

In certain embodiments, the column comprises an inert material.

In certain embodiments, the inert material comprises polymethyl methacrylate (PMMA), polyethylene, polypropylene, polyvinylchloride, and/or silica glass,

In certain embodiments, the column comprises a serpentine column.

In certain embodiments, the one or more target molecules comprise a protein, a carbohydrate, a polynucleotide, a lipid, a vitamin, or an antibiotic. In certain embodiments, the one or more target molecules comprise an antibody.

In certain embodiments, the fluid sample comprises a protein A chromatography eluate comprising an antibody. Antibodies can be eluted from protein A chromatography columns using low pH (e.g., a pH of about 2.0 to about 5.0). In such cases, the protein A chromatography eluate comprising the antibody can be directed or injected into the column without the addition of additional acid. In certain embodiments, the protein A chromatography eluate comprises a pH of about 2.0 to about 5.0. In certain embodiments, the protein A chromatography eluate comprises a pH of 2.0, 2.1, 2.2., 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

In certain embodiments, the second in-line mixer mixes a base solution and the pH-reduced fluid sample, thereby producing a pH-rebalanced fluid sample.

In certain embodiments, the pH-rebalanced fluid sample comprises a pH of about 6.5 to about 8.5. In certain embodiments, the pH-rebalanced fluid sample comprises a pH of about 7.0 to about 8.0. In certain embodiments, the pH-rebalanced fluid sample comprises a pH of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

In one aspect, the disclosure provides a method for inactivating one or more viruses in a fluid sample comprising a target molecule in a column-based continuous viral inactivation system, wherein the method comprises: a) mixing the fluid sample with an acid to lower pH in a first mixing unit; b) continuously transferring the fluid sample from step a) to an inert glass-packed column for incubation at a specified residence time to inactivate viruses; c) periodically adding a protein-free injection solution to the fluid sample before the fluid sample is transferred to the column; d) continuously transferring the fluid sample from step b) to a second mixing unit; e) mixing the fluid sample with a base in the second mixing unit; f) using pH electrodes to measure the pH of the fluid sample after the fluid sample flows through the first and second mixing units, wherein the pH electrodes provide feedback to control acid and base flow; g) monitoring fluid pulse before and after the fluid sample flows through the column by measuring UV absorbance at 280 nm, wherein the monitoring characterizes the residence time distribution of the column in real-time; and h) adjusting total fluid flow in the column-based continuous viral inactivation system to maintain minimum residence time; wherein the viruses are inactivated.

In certain embodiments, the residence time distribution (RTD) is estimated periodically during operation through inverse tracer experiments and used to estimate minimum residence time (MRT), which in turn is used to adjust feed flow rates.

### EXAMPLES

Materials and Methods

Viral Inactivation System

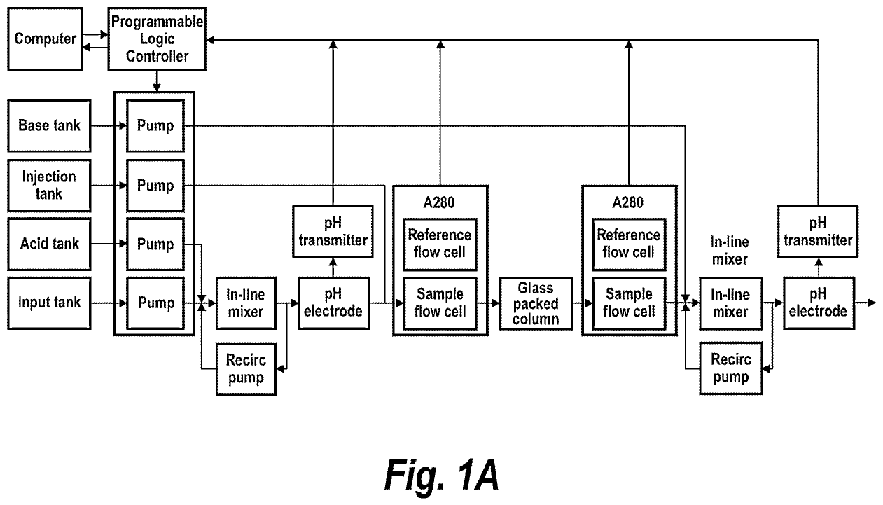

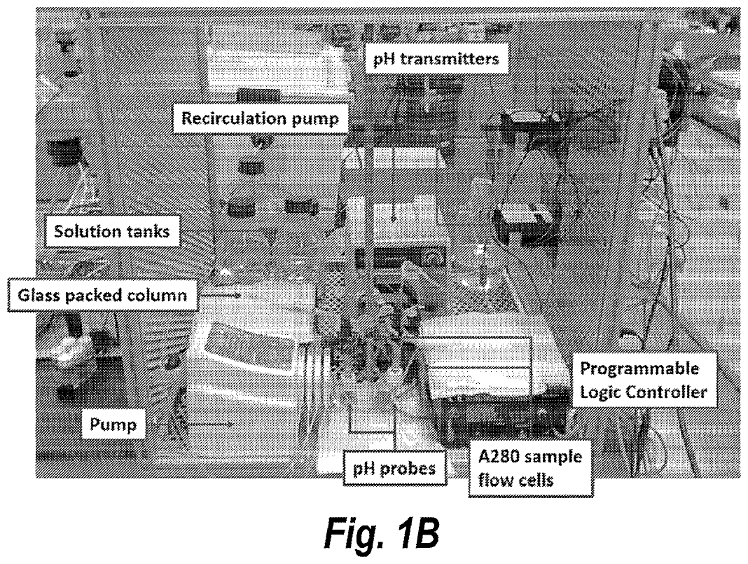

A lab-scale continuous viral inactivation system was constructed. See FIG. 1A and FIG. 1B. A multi-channel peristaltic pump (Ismatech Reglo ICC, 12 roller, 4 channel, 0.51 mm ID Pharmed BPT tubing) was used to pump input solution and acid to a mixing unit comprised of an in-line mixer (Stamixco Model HT) and a peristaltic pump (Masterflex L/S 14, C-Flex ULTRA tubing). The mixed stream flows into an in-line pH electrode (Cole Parmer, Sealed, 800 μL volume), and into a column (Diba Omnifit EZ, 6.6 mm diameter, 250 mm length) packed with inert glass (Sigma-Aldrich, Glass beads, 75 μm). An injection system is used to periodically inject UV transparent solution into the flow stream, with UV absorbance sensors (Spectrum Labs, UV Model 280) at both the column inlet and outlet to measure column transit. The pH of the fluid leaving the column is raised by mixing with base using a second mixing unit and pH electrode.

FIG. 1A illustrates a schematic of the column-based viral inactivation unit. Black lines indicate fluid flow, while red lines indicate information flow. Input solution containing biologics is mixed with acid to lower pH. This fluid is then directed to an inert glass-packed column for incubation at a specified residence time to inactivate viruses. This fluid is then mixed with base in a second mixing unit. pH electrodes are installed after each mixing unit to provide feedback to control acid and base flow. A protein-free injection solution is periodically added before the column. The fluid pulse is monitored before and after the column using UV absorbance at 280 nm and used to characterize the residence time distribution of the column in real-time. Total fluid flow to the system is adjusted to maintain minimum residence time for viral inactivation assurance. FIG. 1B illustrates a photo of column-based viral inactivation unit. Key components in the prototype are labeled. All components were commercial off-the-shelf.

A programmable logic controller (PLC, Koyo Click C0-11DD1E-D) was used for low-level control of the system. The multi-channel peristaltic pump was connected and actuated using the RS-232 interface. The pH transmitter (Cole Parmer, Model 350) was connected using 4-20 mA current loop (Koyo Click C0-04AD-1), while the absorbance sensor was connected using an analog voltage sensor (Koyo Click C0-04AD-2). The PLC was connected via MODBUS TCP to a touch-screen tablet (Microsoft Surface Go MCZ-00001) for data collection, human-machine interface, and online parameter estimation.

A model-based, reaction-invariant controller with maximum a posteriori (MAP) parameter estimation was used for pH control [18]. MAP estimation was performed on the tablet (custom code running in MATLAB 2019a), and parameter estimates forwarded to the PLC to improve the performance and robustness of the reaction-invariant controller. Real-time RTD parameter estimation and MRT estimation was also performed on the attached computer for RTD monitoring and control, and the results forwarded to the PLC for closed-loop control of MRT.

Controller Testing

100 mM sodium hydroxide with 1% (v/v) acetone was used as the test input solution. 1 M phosphoric acid was used as the acid, and 1 M sodium hydroxide was used as the base. The low-absorbance injection solution was deionized water (DIW). The acid was replaced with 50 mM citric acid for the pH controller disturbance testing. A 30 cm loop of 1/16” ID tubing was added to the front of the column using two tees and pinch clamps was used to remove the loop from the flow path during operation for RTD controller disturbance testing.

Viral Clearance Testing

100 mM phosphate, pH 6.5 with 0.05 g/L riboflavin and 1×10⁷ pfu/mL Phi6 bacteriophage was used as the test input solution.

Phi6 bacteriophage and host bacteria, Pseudomonas syringae, were used. Phi6 bacteriophage was grown in P. syringae (1 mL of overnight culture) by overlaying 35 mL of LB agar (10 g/L tryptone, 5 g/L yeast extract, 5 g/L NaCl, and 15 g/L agar) with 6 mL of diluted soft agar (3 mL LB broth and 3 mL LB agar) overnight at 25° C. The Phi6 virus particles were eluted from the top agar with 2 mL of buffer (40 mM Tris base, 150 mM NaCl, and 10 mM MgCl-6H₂O) and concentrated by ultracentrifugation at 14,000 rpm for 15 min at 4° C. The pellet was resuspended in Phi6 assay buffer (21 mM Na₂HPO₄, 11 mM KH₂PO₄, 43 mM NaCl, 9.3 mM NH₄Cl, 0.4 mM MgSO₄, pH 6.5) and passed through 0.2 μm cellulose acetate filter (VWR). Bovine Serum Albumin (BSA) was added to the purified Phi6 to a final concentration of 0.5% (v/v), then stored in −80° C.

Plaque forming assay was used to determine the Phi6 titers. Serial 1:10 dilution of samples was performed 5 times in Phi6 assay buffer. 100 μL of diluted samples or 1 mL of samples were added into 1 mL of host cells and incubated at 25° C. for 5 min. The mixture was added into 3 mL of LB broth and 3 mL of LB agar, overlaid on LB agar plate, and incubated overnight at 25° C.

Results and Discussions

Modeling and Control of pH

In the inactivation process, fluid is first brought to a low-pH state. Fast and accurate control of pH provides rapid inactivation while minimizing impact to product quality. A model-based, reaction-invariant controller was thus used to account for the nonlinearities present in a typical buffer titration curve. To maintain constant total flow rate through the column, the controller manipulated flow ratio instead of flow rate. The pH control system also addresses changing product and buffer concentration due to natural or unexpected variation in bioreactor or capture column operation. MAP estimation was used to address these uncertainties by updating solution pKa and concentrations in response to sensor data. For implementation details, the reader is referred to reference [18], hereby incorporated by reference.

Design of Column-Based Inactivation Reactor

A packed-bed column-based system was chosen due to its narrow residence time distribution and that there is substantial industrial experience in packing and qualifying columns to ensure uniformity. The system is constructed from commercial off-the-shelf components, with glass beads selected for their inert nature and low risk of leaching.

In a column-based design, competing objectives of desired throughput and residence time, low pressure drop, and small dispersion must be simultaneously balanced. The design variables are particle size dₚ, column diameter d and length L, and superficial velocity νₛ. Volumetric flow rate [symbol] and average residence time [symbol] are described by design variables as

$$Q = \frac{π {d}^{2} {υ}_{s}}{4} , τ = \frac{L}{u} = \frac{ϵ L}{{υ}_{s}} , \qquad ( 1 )$$

where μ is the mean velocity and ε is the column porosity. Therefore, column diameter and length are chosen based on the desired throughput and residence time:

$$d = \sqrt{\frac{AQ}{π {υ}_{s}}} and L = \frac{τ {υ}_{s}}{ε} .$$

The pressure drop, as expressed by the Carman-Kozeny equation, can be reduced to

$$Δ P = \frac{180 μ L}{{ϕ}_{s}^{2} {d}_{p}^{2}} \frac{{( 1 - ϵ )}^{2}}{{ϵ}^{3}} {υ}_{s} = \frac{180 μ {τ ( 1 - ϵ )}^{2}}{{ϕ}_{s}^{2} {ϵ}^{4}} {( \frac{{υ}_{s}}{{d}_{p}} )}^{2} , \qquad ( 2 )$$

where μ is the fluid viscosity, and φₛ, is the particle sphericity.

In the same way, the standard deviation of a dispersed peak can be characterized as

$$σ = \sqrt{2 \frac{DL}{{u}^{3}}} = 2 \sqrt{τ \frac{{d}_{p}}{{υ}_{s}}} , \qquad ( 3 )$$

where dispersion coefficient is given by

$$D = \frac{2 {ud}_{p}}{ϵ}$$

(Levenspiel, O. 1999. Chemical Reaction Engineering. 3” ^{d} Edition). The right-hand sides of Equations (2) and (3) indicate that changing the particle size or superficial velocity to reduce the pressure drop increases the dispersion, and vice versa. Therefore, their ratio is chosen to minimize the dispersion while satisfying the pressure limit of the peristaltic pump. In the interest of system compactness, small particle size and superficial velocity are chosen considering the availability of columns and glass beads.

Modeling of RTD

The low-pH solution is incubated for a sufficient time for viral inactivation. At the same time, extended incubation will result in aggregation and acidic species formation, degrading product quality. A quantitative understanding of the process allows balancing between these two competing effects. This comes in the form of an RTD, which describes the amount of time that a fluid element spends within the system by a probability distribution function E.

The column can be modeled as a plug flow reactor (PFR) with axial dispersion in series with a continuously stirred tank reactor (CSTR), with the respective RTDs

$${E}_{PFR} ( θ , Pe , z ) = \sqrt{\frac{Pe}{4 π x θ}} exp ( - \frac{{Pe ( x - θ )}^{2}}{4 x θ} ) , \qquad ( 4 )$$

$${E}_{CSTR} ( θ , x ) = \frac{1}{1 - x} exp ( - \frac{θ}{1 - x} ) . \qquad ( 5 )$$

where Pe is the Peclet number, x is the residence time fraction of PFR, and θ(t,τ) is the dimensionless time. The concentration of any component at the outlet of the column can be calculated through the convolution

Cₒᵤₜ(θ)=Cᵢₙ(θ)*E(θ,Pe,x)=∫₀^{θ}Cᵢₙ(θ−θ′)E(θ′,Pe,x)dθ′, (6)

E(θ,Pe,x)=E_{PFR}(θ,Pe,x)*E_{CSTR}(θ,x)=∫₀^{θ}E_{PFR}(θ−θ′,Pe,x)E_{CST}(θ′,x)dθ′. (7)

The cumulative distribution function F can be computed as

F(θ,Pe,x)=∫₀^{θ}E(θ′,Pe,x)dθ′. (8)

which in turn allows us to define the minimum residence time τₘᵢₙ as

F(θ(τₘᵢₙ,τ),Pe,x)=η, (9)

where η is a small value corresponding to the fraction of material leaving before the minimum residence time.

Control of Residence Time

RTD is affected by variables such as reactor design, geometry, fouling, pump drift, and input flow rate. These variables can be classified into three types. Manipulated variables are those that can be controlled online, like input flow rates. Disturbance variables are those that may vary during operation, but cannot be effectively controlled, like fouling or pump drift. Design variables are those that can only be controlled offline, like reactor design and geometry.

While design variables have been explored quite extensively by work such as references [11-15], the topic of disturbance rejection has not been discussed directly. Disturbance rejection is even more important as these continuous reactors are deployed in pilot or commercial scale and operated for extended period than lab-scale systems.

It is impossible to completely reject the effect of disturbances on the RTD; RTD is infinite-dimensional and cannot be controlled with scalar manipulated variables like flow rates. It is however possible to reject disturbances on a scalarization of the RTD. The LRV approach, where a nonlinear model is used to relate the residence time distribution to a scalar LRV, is one of the scalarization. Alternatively, the RTD can be scalarized by the MRT approach, where an MRT is defined as the scalar time before which only a small fraction of material leaves. This fraction varies from 0.005 [11-13] to 10⁻⁵ [15]. Alternatively, the MRT has been defined by subtracting five standard deviations of the RTD from the average residence time (Brown & Orozco, Biotechnology & Bioengineering. 2021). While the LRV approach allows for direct addressing of the critical quality attribute (CQA), it relies on an accurate inactivation model, which may vary from virus to virus and thus a priori uncertain. This disclosure avoids this uncertainty by utilizing the 0.005 fraction MRT approach, while at the same time noting that the 10⁻⁵ MRT or LRV approach can be trivially implemented given, in the latter case, known inactivation kinetics.

MRT control includes three components: (1) a means of measuring RTD/MRT, (2) a feedback control algorithm for adjusting flow rate in response to MRT, and (3) a means of actuating flow rate.

Measurement of RTD and MRT

At the low values of q, direct measurement of MRT using tracer experiments is impossible due to in-line sensor noise obscuring observability. To overcome lack of observability, RTD can be estimated as a parametric distribution, which can in turn be used to determine MRT.

Using the Beer-Lambert law, the absorbance A₂₈₀(t)∝C(t) can be written as

A_{280,out}(θ)=A_{280,in}(θ)*E(θ,Pe,x). (10)

The parameters τ, Pe, and x can be calculated from the absorbance measurement through the solution of the sum-of-squares minimization problem:

$${arg max}_{τ , Pe , x} {\int }_{0}^{T} {[ {A}_{280 , in} ( θ ( t , τ ) ) * E ( θ ( t , τ ) , Pe , x ) - {A}_{280 , out} ( θ ( t , τ ) ) ]}^{2} dt , \qquad ( 11 )$$

and used to estimate MRT through Equations (7) and (8). PGP-39X

Sufficient variability in A_{280,in} allows for accurate parameter estimation. This can be achieved through normal process variation, such as concentration peaks inherent in multi-column chromatography elution. In the case that the system is operated from a large, well-mixed tank and is devoid of variation, concentration variation is deliberately introduced.

This is safely achieved through the periodic injection of protein-free buffer, which has negligible UV absorbance relative to that of protein-laden input solution. This effectively dilutes the solution, lowering the absorbance, and providing the desired variability.

Feedback Control Algorithm and Actuation

Given the MRT estimation, a feedback control algorithm

$${ω}_{i + 1} = \frac{{ω}_{i} {τ}_{min , i}}{{τ}_{min , SP}} , \qquad ( 12 )$$

where ω is the pump rotational speed and the subscripts i refers to the current time step, i+1 refers to the next time step, and SP refers to the setpoint, can be used to provide a new pump speed.

Since peristaltic pump flow rates are almost linear to rotational speed, the system can attain the setpoint within a single step after an RTD/MRT measurement. Any nonlinearity or disturbance (e.g. tubing stretch) is also addressed through feedback control.

A consideration, however, is that having feed flow rate used for residence time control removes a degree of freedom in responding to changes in upstream flow into the unit. This can be solved by slightly diluting the product stream or introducing a surge tank.

Controller Validation Experiments

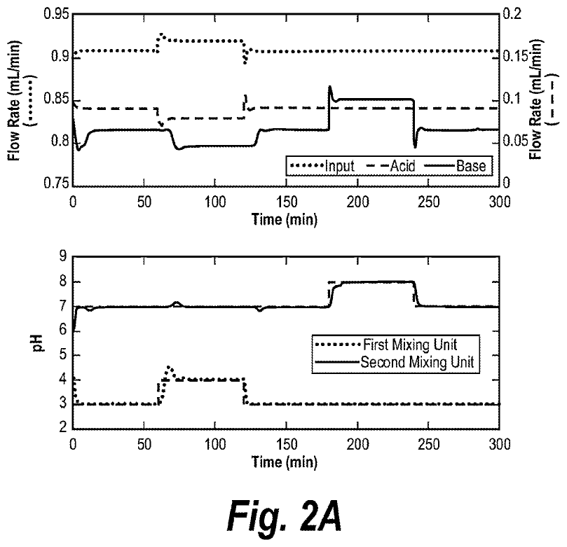

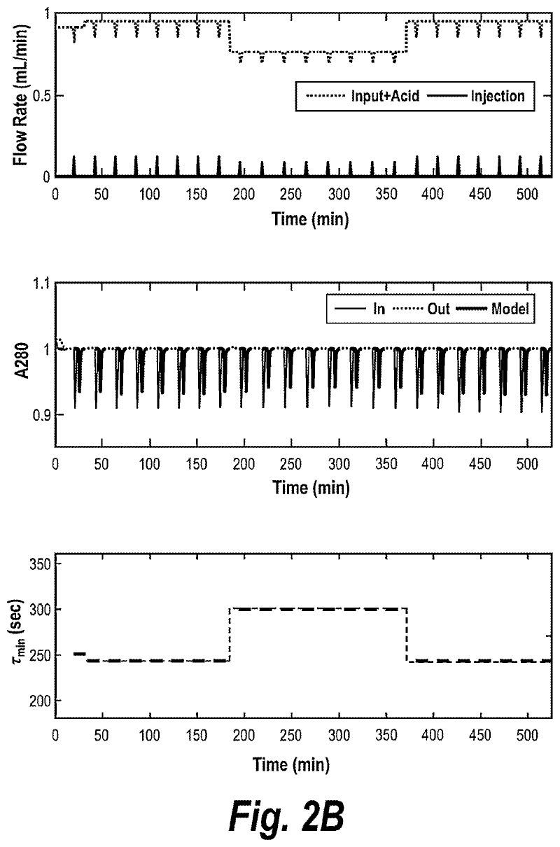

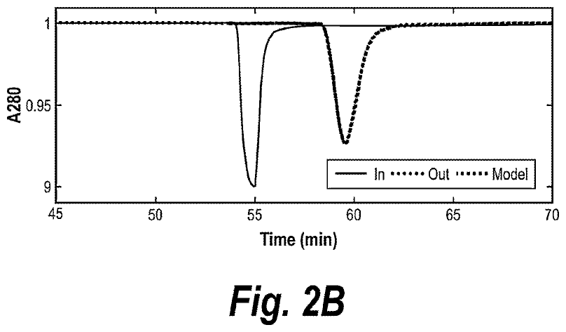

Setpoint tracking was tested for the pH controllers, showing fast and accurate response, with no instability, while following a step up and down of pH (FIG. 2A). Setpoint tracking was also tested for the MRT controller following a step up and down of MRT, again showing fast and accurate response with no instability (FIG. 2B).

FIG. 2A illustrates results of the pH controller setpoint tracking test. pH setpoint for the first mixing unit was changed from 3 to 4 at 60 minutes and reverted to 3 at 120 minutes. pH setpoint for second mixing unit was changed from 7 to 8 at 180 minutes and reverted to 7 at 240 minutes. Fast and accurate response was observed with no instability.

FIG. 2B illustrates results of the MRT controller setpoint tracking test. MRT was changed from 240 seconds to 300 seconds at 180 minutes and reverted to 240 seconds at 360 minutes. Fast and accurate response was observed with no instability. FIG. 2C illustrates results of absorbance measurements at the inlet and outlet of the column, overlaid with the RTD model.

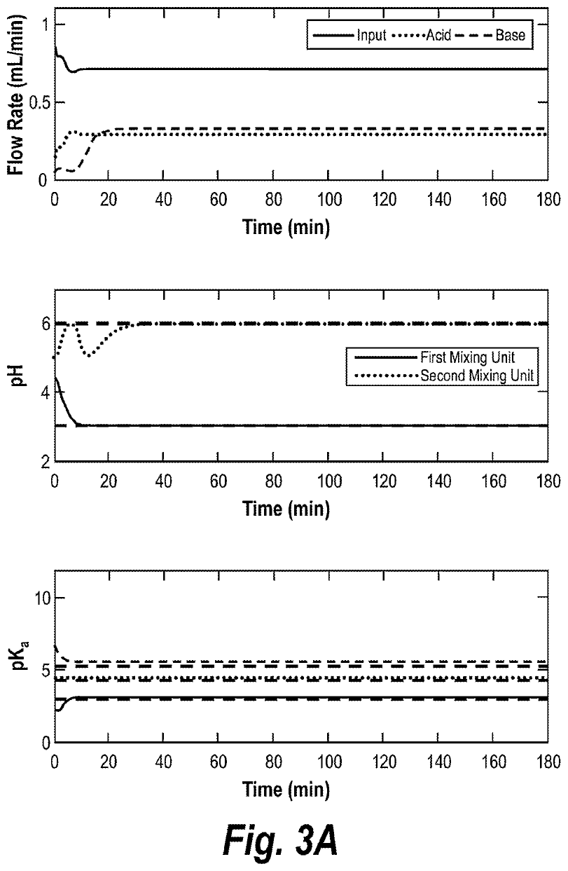

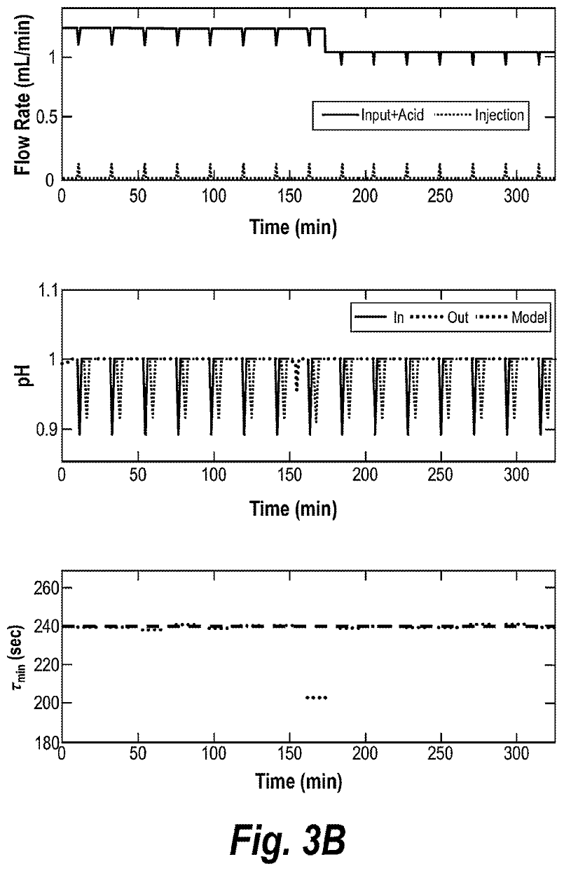

pH disturbance rejection was tested by changing the buffer of the acid stream used within the system from phosphoric acid to citric acid. The system not only remains stable, but correctly estimates the pKₐ of citric acid purely from the data obtained from the sensors (FIG. 3A).

FIG. 3A illustrates results of the pH controller disturbance rejection test. System was started with citric acid instead of phosphoric acid despite the model being initialized with the phosphoric acid parameters. The MAP estimation algorithm correctly estimated the new pKa. Fast and accurate response was observed with no instability.

MRT disturbance rejection was tested by adding an additional length of tubing in series with the column. While the system is in normal operation, tube clamps were used to remove the additional length of tubing from the flow path, effectively subtracting dead volume and changing the system RTD. Despite this sudden change, the RTD is correctly measured and the MRT correctly re-estimated (FIG. 3B). The controller adjusts the pump speeds immediately after receiving the new MRT to maintain the MRT setpoint (FIG. 3B).

FIG. 3B illustrates results of the MRT controller disturbance rejection test. The system was first started with an additional dead volume in front of the column. At 160 minutes, the dead volume was bypassed, reducing system volume. The MRT measurement system correctly identifies the change in residence time and adjusts pump speed, restoring the original residence time setpoint in a single step. Model correctly predicts the output of each water injection.

Viral Inactivation—pH

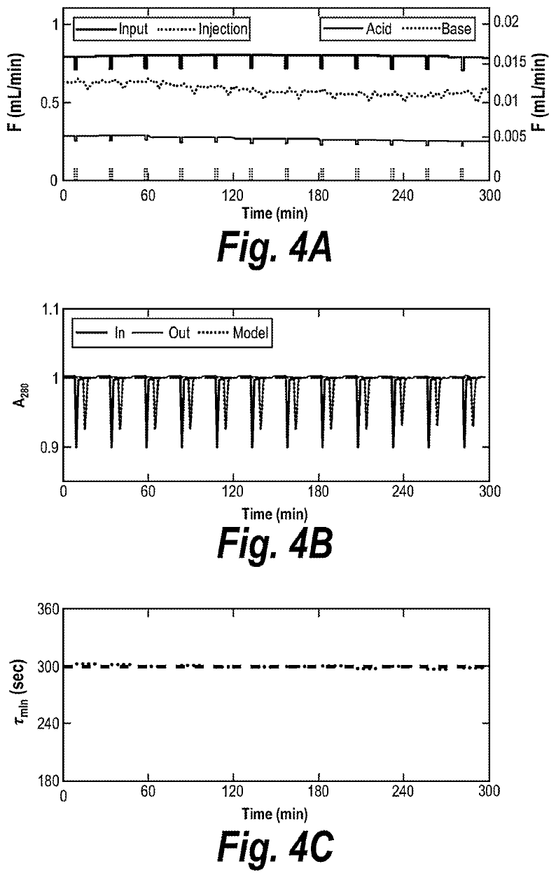

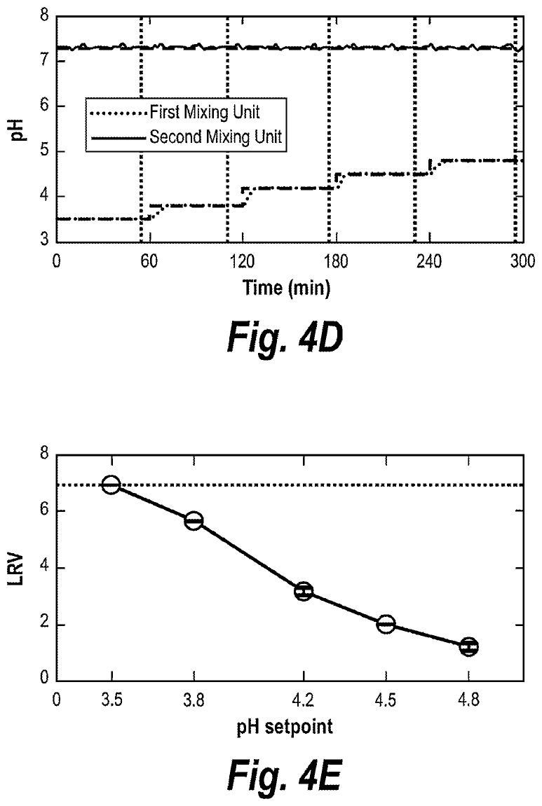

Viral inactivation was demonstrated by varying the pH setpoints from 3.5 to 4.8 at an MRT setpoint of 5 minutes (FIG. 4A-FIG. 4E). The Phi6 bacteriophage was below the limit of detection at pH 3.5 and inactivation kinetics were slower at the higher pH setpoints.

The system was sampled at pH 3.5, 3.8, 4.2, 4.5, and 4.8. The pH setpoint for the second mixing unit was 7.3 and MRT setpoint was 5 minutes. As shown in FIG. 4E, the logarithmic reduction values (LRVs) decreased as the pH setpoint of the first mixing unit was increased. The horizontal dotted line shows the limit of detection (no live virus was detected for values on the horizontal dotted line). Accordingly, the virus was effectively inactivated at all pH values tested.

Viral Inactivation—MRT

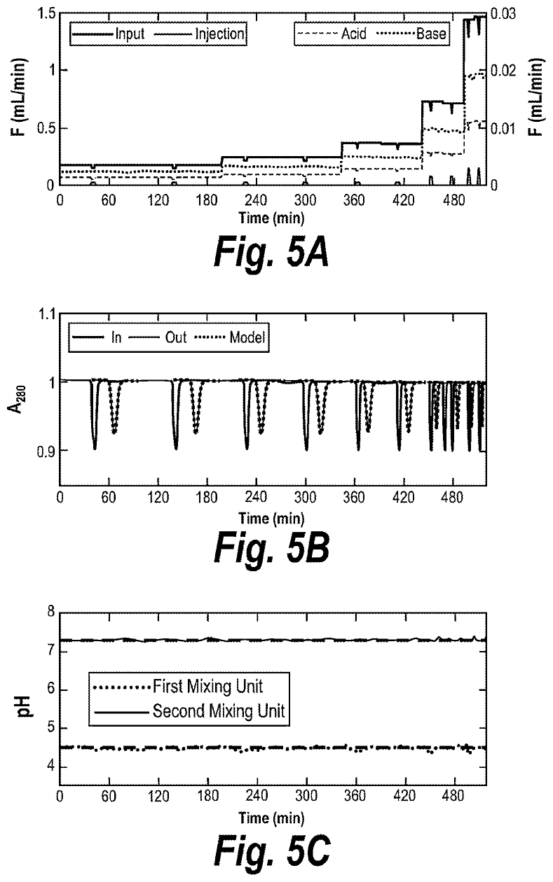

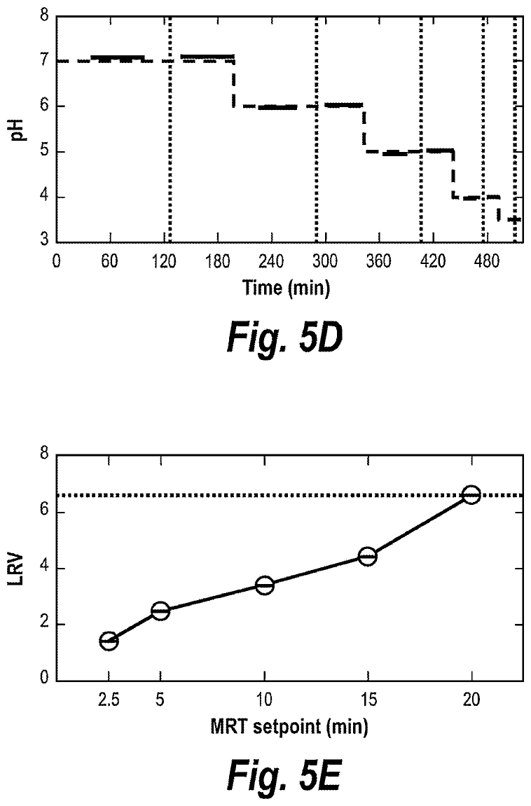

Viral inactivation was also tested with varying MRT setpoints from 20 minutes to 2.5 minutes at a pH setpoint of 4.5 (FIG. 5A-FIG. 5E). The Phi6 bacteriophage was below the limit of detection at a MRT of 20 minutes and the LRV values decreased at the lower MRT setpoints.

The MRT setpoint was changed from 20 minutes to 15, 10, 5, and 2.5 minutes. The pH setpoint for the first mixing unit was 4.5 and the pH setpoint for the second mixing unit was 7.3. As shown in FIG. 5E, the LRVs decreased as the MRT setpoint decreased. The horizontal dotted line shows the limit of detection (no live virus was detected for values on the horizontal dotted line). Accordingly, the virus was effectively inactivated at all MRT values tested.

Viral Inactivation—pH 4.5/MRT 15 Minutes

Reproducibility of viral inactivation was tested at fixed setpoints of pH 4.5 for the first mixing unit and MRT 15 minutes for about 15 hours. A pH setpoint for the second mixing unit was used. The system resulted in stable control of both pH and MRT during the operation. Collected samples during the operation also showed a consistent LRV value with a standard deviation of 0.08, which is smaller than standard deviations of triplicate analysis of each sample 0.12.

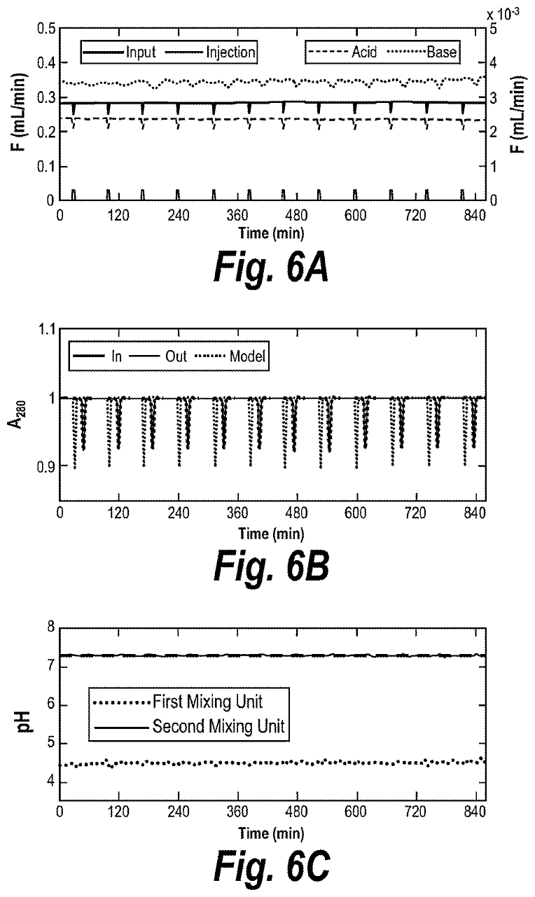

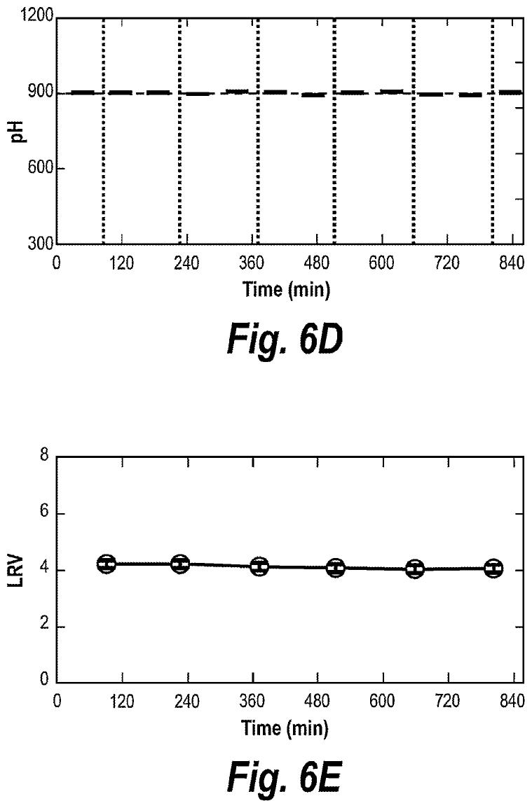

As shown in FIG. 6E, the LRVs of the samples were sufficiently low at various operating times.

Alternative Strategies For Controlling Changing Inlet/Input Flow Rates

Strategy 1—Holding Tank

A holding tank can be placed upstream of the viral inactivation system, with the same control system described above being implemented. The size of the holding tank is specified by the amount of variation in the flow rate from the upstream process over time. A small holding tank can be used when the amount of variation in the flow rate from the upstream process is small and the column is appropriately sized.

Strategy 2—Changing pH Setpoint

The pH setpoint can be changed in response to the inlet/input solution flow rate specified/determined by the upstream process. The pH setpoint can be adjusted such that the specified viral inactivation is ensured to occur in spite of the change in residence time. For example, but in no way limiting, the reduction value (RV) and logarithmic reduction value (LRV) are given by (these relationships are specified by the viral inactivation kinetics):

$$RV ( t ; pH ) = \frac{C ( t ; pH )}{{C}_{0}} = {10}^{- k ( {pH}_{0} - pH ) t} ,$$

$$LRV ( t ; pH ) = - {log}_{10} RV ( t ; pH ) = k ( {pH}_{0} - pH ) t ,$$

where k is a proportional constant and pH₀ would correspond to a pH in which the viral inactivation is very low, with pH≤pH₀. Then the above expression can be inserted into the lower bound for LRV in the system (η is a scalar between 0 and 1), to give the inequality

LRV_{system}≥(1−η)k(pH₀−pH)τₘᵢₙ.

The periodic (or aperiodic) estimation of the residence time distribution (RTD) and minimum residence time (MRT) can be used to compute the product of the total flow rate F through the column and the minimum residence time, ν=Fτₘᵢₙ. Then the pH setpoint can be adjusted based on the updated ν and choice of LRV setpoint:

$${LRV}_{SP} = \frac{( 1 - η ) k ( {pH}_{0} - {pH}_{SP} ( t ) ) v}{F ( t )} ,$$

$${pH}_{SP} ( t ) = {pH}_{0} - \frac{{LRV}_{SP} F ( t )}{( 1 - η ) kv} .$$

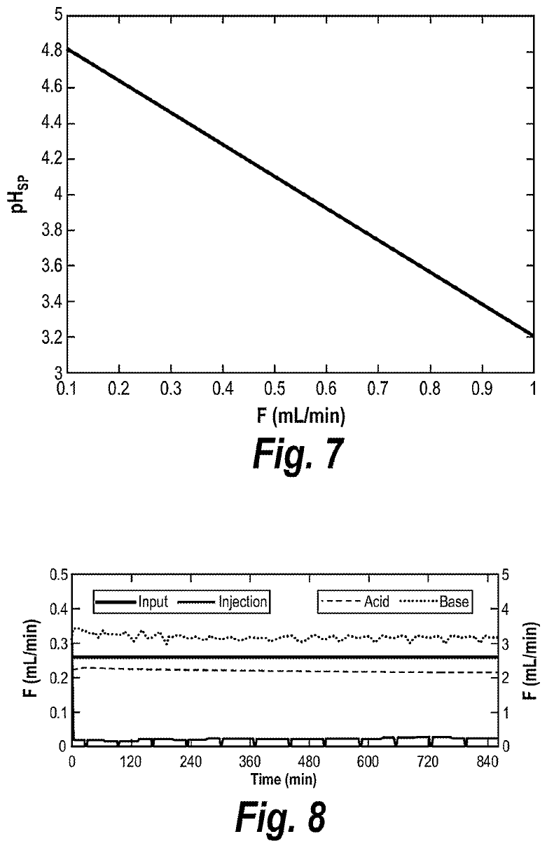

This equation updates the pH setpoint to compensate for variation in the total flow rate with time. FIG. 7 is a plot showing how the pH setpoint would be changed with respect to the total flow rate through the column for LRV_{SP}=5, n=0.005, ν=4 [mL], pH₀=5.0, and

$$k = 0.7 [ \frac{1}{min} ] ,$$

where the parameters ν, pH₀, and k are obtained from experimental data in FIG. 4-FIG. 6 of the disclosure.

Strategy 3—Dilute Stream To Increase Flow Rate

In this disclosure, the overall feed flowrate (inlet/input solution+acid+DIW) is manipulated to control the minimum residence time, and the ratio of the inlet/input solution and the acid flowrates is manipulated for control of pH. For example, in the plot show in FIG. 6A, DIW is injected periodically and the inlet/input solution and acid flow rates were reduced at the same time so that the total of the three flow rates is a constant (the base flow after the outlet of the column is set to whatever is needed to achieve the desired pH at that point, and does not affect the residence time distribution of the column).

In a situation in which the inlet/input solution comes directly from an upstream process (e.g., a protein A column eluate), the inlet/input solution flowrate is specified/determined by the upstream process. The same control system described above can be implemented by selecting the size of the column in the viral inactivation system to be large enough to be able to achieve the desired minimum residence time for the maximum potential value for the inlet/input flow rate and whatever acid flow rate is needed for pH control and whatever DIW flow would be injected for the RTD measurement. Then the desired minimum residence time can be achieved for lower values of the inlet/input flow rate as well, by injecting additional DIW beyond what was used above as a negative tracer. The additional DIW water can be injected using the same system used to inject DIW for the RTD measurement. Alternatively, a different injection system could be used.

One instantiation of this design is to measure the RTD by using a brief temporary reduction of the DIW flow described above, and use this reduction in the DIW flow to measure the RTD. In this design, the residence time would be slightly reduced temporarily, which would still satisfy the minimum residence time requirement, but would slightly increase the residence time to larger than needed to meet that requirement.

For a small range of feed flow variations from an upstream process, the additional DIW addition will only slightly dilute the feed stream while maintaining tight control of the residence time. If the range of allowable feed flow variations from the upstream process is larger, then the range of additional DIW injected to achieve the desired control of residence time will be larger, since this design specifies the total flow into the column to be constant or nearly constant. The additional DIW can be injected at each point in time to make up for any reduction in the flow rate from the upstream process. This same approach works if the DIW is replaced by an alternative liquid solution, whether being used to cancel out variations in the flow from the upstream process or being used as an inverse tracer. The same approach also works irrespective of the method in which the DIW is fed to the stream (e.g., by a T, Y, or a needle). The DIW used for the inverse tracer could be replaced by any tracer or inverse tracer. The solution used to make up for variation in the feed stream sent to the system can be replaced by an alternative liquid solution other than DIW. The liquid solutions used for the RTD measurement and for the make-up solution can be different, and it can be beneficial to have these solutions have different compositions, e.g., to increase the signal-to-noise ratio for the RTD measurement, or to reduce the effect of the addition of the make-up solution, e.g., to increase protein stability.

For example, but in no way limiting, consider the implementation of the above approach for a column desired to be large enough to handle the inlet/input flowrate shown in FIG. 6A. The plot of FIG. 8 shows flow rates chosen by the controller for an extended operation to confirm the repeatability of the viral inactivation for the alternative inlet/input flowrate fixed by the upstream process to be 0.26 mL/min. In this example, instead of introducing DIW as an inverse tracer periodically for the RTD measurement, the DIW dilution stream used for the minimum residence time control was stopped periodically for the RTD measurement. This strategy leads to a small residence time increase during this period due to slight decrease of overall flow rate through the column, but since this dilute stream is small compared to the overall flowrate, this increase has a negligible effect on the product while still guaranteeing viral inactivation.

### CONCLUSIONS

This disclosure describes a low-cost, column-based, continuous-flow viral inactivation system constructed with off-the-shelf components that provides tight control of the operating pH and minimum residence time (MRT). The system injected acid solution into the feed stream using a mixing unit whose outlet flows to an in-line pH electrode. pH measurements were used to adjust the flow rates to control the pH based on a model-based reaction-invariant controller that uses Bayesian estimation to ensure robustness. The mixed stream then flowed into a column that has UV absorbance sensors at the inlet and outlet. UV-transparent inverse tracer was periodically injected into the flow stream to estimate the residence time distribution (RTD) and MRT, which was used to adjust feed flow rates to control the MRT based on a feedback control algorithm. The pH of the fluid leaving the column was raised by mixing with base solution whose flow rate was specified by the model-based controller.

Controller validation experiments for step changes in the setpoints and sudden disturbances demonstrated tight control of the pH and MRT. Bayesian estimation for pH control correctly estimated the pKₐ values from the pH measurements and the RTD was correctly estimated from the absorbance measurements, which enabled stable control of both the pH and MRT, even with sudden disturbances. Viral inactivation experiments demonstrated the connection between the critical process parameters of operating pH and MRT and the critical quality attribute of logarithmic reduction value (LRV) and tight control of both over extended operation.

The capability of the system to tightly control critical process parameters enabled continuous operation of viral inactivation at optimal conditions not only for viral inactivation but also for product quality by preventing over-incubation or excessive pH adjustment. Such a capability can contribute to increasing productivity, improving product quality, and enhancing patient safety of the continuous manufacturing of biologics.

It should be understood that the subject matter defined in the appended claims is not necessarily limited to the specific implementations described above. The specific implementations described above are disclosed as examples only.

### BIBLIOGRAPHY

[1] L. E. Crowell, A. E. Lu, K. R. Love, A. Stockdale, S. M. Timmick, D. Wu, Y. A. Wang, W. Doherty, A. Bonnyman, N. Vecchiarello, et al., “On-demand manufacturing of clinical-quality biopharmaceuticals,” Nature Biotechnology, vol. 36, pp. 988-995, 2018.
[2] K. B. Konstantinov and C. L. Cooney, “White paper on continuous bioprocessing. May 20-21, 2014 continuous manufacturing symposium,” Journal of Pharmaceutical Sciences, vol. 104, no. 3, pp. 813-820, 2015.
[3] A. E. Lu, J. A. Paulson, N. J. Mozdzierz, A. Stockdale, A. N. F. Versypt, K. R. Love, J. C. Love, and R. D. Braatz, “Control systems technology in the advanced manufacturing of biologic drugs,” in 2015 IEEE Conference on Control Applications (CCA), pp. 1505-1515, IEEE, 2015.
[4] M. S. Hong, K. A. Severson, M. Jiang, A. E. Lu, J. C. Love, and R. D. Braatz, “Challenges and opportunities in biopharmaceutical manufacturing control,” Computers & Chemical Engineering, vol. 110, pp. 106-114, 2018.
[5] I. H. T. Guideline, “Viral safety evaluation of biotechnology products derived from cell lines of human or animal origin q5a (r1),” 1997.
[6] W. H. Organization et al., “Guidelines on viral inactivation and removal procedures intended to assure the viral safety of human blood plasma products,” WHO Technical Report, Series, vol. 924, pp. 150-224, 2004.
[7] G. Miesegaes, S. Lute, and K. Brorson, “Analysis of viral clearance unit operations for monoclonal antibodies,” Biotechnology and Bioengineering, vol. 106, no. 2, pp. 238-246, 2010.
[8] A. A. Shukla, B. Hubbard, T. Tressel, S. Guhan, and D. Low, “Downstream processing of monoclonal antibodies—application of platform approaches,” Journal of Chromatography B, vol. 848, no. 1, pp. 28-39, 2007.
[9] G. Sofer, “Virus inactivation in the 1990s—and into the 21st century,” Biopharm international, vol. 16, pp. 50-57, 2003.
[10] Pall Life Sciences, “Application note: Cadence virus inactivation system,” 2018.
[11] S. Klutz, M. Lobedann, C. Bramsiepe, and G. Schembecker, “Continuous viral inactivation at low pH value in antibody manufacturing,” Chemical Engineering and Processing: Process Intensification, vol. 102, pp. 88-101, 2016.
[12] R. Orozco, S. Godfrey, J. Coman, L. Amarikwa, S. Parker, L. Hemandez, C. Wachuku, B. Mai, B. Song, S. Hoskatti, et al., “Design, construction, and optimization of a novel, modular, and scalable incubation chamber for continuous viral inactivation,” Biotechnology progress, vol. 33, no. 4, pp. 954-965, 2017.
[13] S. A. Parker, L. Amarikwa, K. Vehar, R. Orozco, S. Godfrey, J. Coffman, P. Shamlou, and C. L. Bardliving, “Design of a novel continuous flow reactor for low pH viral inactivation,” Biotechnology and bioengineering, vol. 115, no. 3, pp. 606-616, 2018.
[14] C. Gillespie, M. Holstein, L. Mullin, K. Cotoni, R. Tuccelli, J. Caulmare, and P. Greenhalgh, “Continuous in-line virus inactivation for next generation bioprocessing,” Biotechnology journal, vol. 14, no. 2, p. 1700718, 2019.
[15] L. Amarikwa, R. Orozco, M. Brown, and J. Coffman, “Impact of dean vortices on the integrity testing of a continuous viral inactivation reactor,” Biotechnology journal, vol. 14, no. 2, p. 1700726, 2019.
[16] D. L. Martins, J. Sencar, N. Hammerschmidt, B. Tille, J. Kinderman, T. R. Kreil, and A. Jungbauer, “Continuous solvent/detergent virus inactivation using a packed-bed reactor,” Biotechnology journal, p. 1800646, 2019.
[17] L. Arnold, K. Lee, J. Rucker-Pezzini, and J. H. Lee, “Implementation of fully integrated continuous antibody processing: effects on productivity and COGm,” Biotechnology journal, vol. 14, no. 2, p. 1800061, 2019.
[18] A. E. Lu, J. A. Paulson, and R. D. Braatz, “pH and conductivity control in an integrated biomanufacturing plant,” in 2016 American Control Conference (ACC), pp. 1741-1746, IEEE, 2016.

1. A column-based continuous viral inactivation system, comprising a pH feedback controller to adjust feed flow rates.

2. A column-based continuous viral inactivation system, comprising a minimum residence time (MRT) feedback controller to adjust feed flow rates.

3. A column-based continuous viral inactivation system, comprising a pH feedback controller and a minimum residence time (MRT) feedback controller to adjust feed flow rates.

4. The system of any one of claims 1-3, wherein the feedback controllers estimate residence time distribution (RTD) periodically during operation to adjust feed flow rates.

5. The system of claim 4, wherein the feedback controllers estimate RTD at least once during operation to adjust feed flow rates.

6. The system of claim 4, wherein the feedback controllers estimate RTD 1, 2, 3, 4, 5, 6, 7, 8, 9, 10, or more times during operation to adjust feed flow rates.

7. The system of any one of claims 4-6, wherein the RTD is estimated by injecting a tracer.

8. The system of claim 7, wherein the tracer is UV-transparent.

9. The system of any one of claims 2-8, wherein the MRT is about 1 minute to about 30 minutes.

10. The system of any one of claims 2-9, wherein the MRT is 2.5 minutes, about 4 minutes, about 5 minutes, about 10 minutes, about 15 minutes, or about 20 minutes.

11. The system of any one of claims 1-10, comprising:
a first in-line mixer;
a first pH electrode;
a first UV absorbance sensor;
a column;
a second UV absorbance sensor;
a second in-line mixer; and
a second pH electrode;
wherein each of the first in-line mixer, column, and second in-line mixer comprise an inlet, an outlet, and a tubular flow path.

12. The system of claim 11, wherein the first in-line mixer mixes an acid solution and a fluid sample comprising one or more target molecules, thereby producing a pH-reduced fluid sample.

13. The system of claim 12, wherein the pH-reduced fluid sample comprises a pH of about 2.0 to about 5.0.

14. The system of claim 12 or 13, wherein the pH-reduced fluid sample comprises a pH of 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

15. The system of any one of claims 11-14, wherein the first pH electrode and second pH electrode provide the pH to the pH feedback controller.

16. The system of any one of claims 11-15, wherein the pH-reduced fluid sample is incubated in the column for a time sufficient to inactivate one or more viruses in the pH-reduced fluid sample.

17. The system of any one of claims 11-16, wherein at least about 99.5% of the pH-reduced fluid sample by volume is incubated in the column for about 1 minute to about 120 minutes.

18. The system of any one of claims 11-17, wherein the column comprises an inert material.

19. The system of claim 18, wherein the inert material comprises polymethyl methacrylate (PMMA), polyethylene, polypropylene, polyvinylchloride, and/or silica glass.

20. The system of any one of claims 11-19, wherein the column comprises a serpentine column.

21. The system of any one of claims 11-20, wherein the one or more target molecules comprise a protein, a carbohydrate, a polynucleotide, a lipid, a vitamin, or an antibiotic.

22. The system of any one of claims 11-21, wherein the one or more target molecules comprise an antibody.

23. The system of any one of claims 11-22, wherein the fluid sample comprises a protein A chromatography eluate comprising an antibody.

24. The system of claim 23, wherein the protein A chromatography eluate comprises a pH of about 2.0 to about 5.0.

25. The system of claim 23, wherein the protein A chromatography eluate comprises a pH of 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

26. The system of any one of claims 12-25, wherein the second in-line mixer mixes a base solution and the pH-reduced fluid sample, thereby producing a pH-rebalanced fluid sample.

27. The system of claim 26, wherein the pH-rebalanced fluid sample comprises a pH of about 6.5 to about 8.5.

28. The system of claim 26, wherein the pH-rebalanced fluid sample comprises a pH of about 7.0 to about 8.0.

29. A column-based continuous viral inactivation system, comprising a minimum residence time (MRT) sufficient to inactivate a substantial portion of viruses in a fluid sample, wherein the MRT is maintained by periodically injecting a tracer into the system.

30. The system of claim 29, wherein the MRT comprises a time for about a 0.01 fraction, 0.005 fraction, 0.001 fraction, 0.0005 fraction, 0.0001 fraction, 0.00005, or a 0.00001 fraction of a volume of the fluid sample to leave the system.

31. The system of claim 29, wherein the MRT comprises a time for about a 0.005 fraction of a volume of fluid to leave the system.

32. The system of claim 29, wherein the MRT comprises about 1 minute to about 60 minutes.

33. The system of claim 29, comprising an MRT sufficient to inactivate about 95%, about 96%, about 97%, about 98%, about 99%, about 99.5%, about 99.6%, about 99.7%, about 99.8%, about 99.9%, or 100% of viruses in a fluid sample.

34. The system of claim 29, wherein the tracer injected at least once during operation to maintain the MRT.

35. The system of claim 29, wherein the tracer injected 1, 2, 3, 4, 5, 6, 7, 8, 9, 10, or more times during operation to maintain the MRT.

36. The system of claim 29, wherein the tracer is UV-transparent.

37. The system of claim 29, wherein the tracer is deionized water.

38. A method for inactivating one or more viruses in a fluid sample comprising a target molecule in a column-based continuous viral inactivation system, wherein the method comprises:
a) mixing the fluid sample with an acid to lower pH in a first mixing unit;
b) continuously transferring the fluid sample from step a) to an inert column for incubation at a specified residence time to inactivate viruses;
c) periodically adding a protein-free injection solution to the fluid sample before the fluid sample is transferred to the column;
d) continuously transferring the fluid sample from step b) to a second mixing unit;
e) mixing the fluid sample with a base in the second mixing unit;
f) using pH electrodes to measure the pH of the fluid sample after the fluid sample flows through the first and second mixing units, wherein the pH electrodes provide feedback to control acid and base flow;
g) monitoring fluid pulse before and after the fluid sample flows through the column by measuring UV absorbance at 280 nm, wherein the monitoring characterizes the residence time distribution of the column in real-time; and
h) adjusting total fluid flow in the column-based continuous viral inactivation system to maintain minimum residence time; wherein the viruses are inactivated.

39. The method of claim 29, wherein the residence time distribution (RTD) is estimated periodically during operation through tracer experiments and used to estimate minimum residence time (MRT), which in turn is used to adjust feed flow rates.
